(19) United States
(12) Patent Application Publication
(10) Pub. No.: US 2011/0112507 A1
(43) Pub. Date: May 12, 2011

(54) CURABLE MATERIAL DELIVERY SYSTEMS AND METHODS

(75) Inventors: Evan D. Linderman, Northbrook, IL (US); John A. Krueger, Muskego, WI (US)

(73) Assignee: CAREFUSION 207, INC., San Diego, CA (US)

(21) Appl. No.: 12/615,606

(22) Filed: Nov. 10, 2009

(51) Int. Cl. A61M 31/00; A61M 5/36

(52) U.S. Cl. 604/506; 604/272

(57) ABSTRACT

A distal end of a cannula immediately proximate a target site within bone. A portion of a cavity-forming device is extended through the cannula and distally beyond the distal end, and then operated to form a cavity at the target site. A track is defined in tissue of the target site between the distal end of the cannula and the cavity. The cavity-forming device is removed from the cannula, and replaced with a delivery tube. A distal tip of the delivery tube is directed distally beyond the distal end of the cannula, through the track and into the cavity. Finally, a material (e.g., a curable material) is delivered through the delivery tube and into the cavity. The cannula can remain stationary following initial insertion, and curable material is not directly deposited into the normally occurring “dead space”.

### BACKGROUND

The present disclosure relates to systems and methods for stabilizing bone structures. More particularly, it relates to systems and methods for delivering a curable, stabilizing material into a bone structure such as vertebral body.

Surgical intervention at damaged or compromised bone sites has proven highly beneficial for patients, for example patients with back pain associated with vertebral damage.

Bones of the human skeletal system include mineralized tissue that can be generally categorized into two morphological groups: “cortical” bone and “cancellous” bone. Outer walls of all bones are composed of cortical bone, which has a dense, compact bone structure characterized by a microscopic porosity. Cancellous or “trabecular” bone forms the interior structure of bones. Cancellous bone is composed of a lattice of interconnected slender rods and plates known by the term “trabeculae.”

During certain bone-related procedures, cancellous bone is supplemented by an injection of a palliative (or curative) material employed to stabilize the trabeculae. For example, superior and inferior vertebrae in the spine can be beneficially stabilized by the injection of an appropriate, curable material (e.g., PMMA or other bone cement or bone curable material). In other procedures, percutaneous injection of stabilization material into vertebral compression fractures, by, for example, transpedicular or parapedicular approaches, has proven beneficial in relieving pain and stabilizing damaged bone sites. Other skeletal bones (e.g., the femur) can be treated in a similar fashion. Regardless, bone in general, and cancellous bone in particular, can be strengthened and stabilized by palliative insertion or injection of bone-compatible material.

Using vertebroplasty as a non-limiting example, a conventional technique for delivering the bone stabilizing material entails placing a cannula with an internal stylet into the targeted delivery site. The cannula and stylet are used in conjunction to pierce the cutaneous layers of a patient above the hard tissue to be supplemented, then to penetrate the hard cortical bone of the vertebra, and finally to traverse into the softer cancellous bone underlying the cortical bone. Once positioned in the cancellous bone, the stylet is then removed, leaving the cannula in the appropriate position for delivery of curable material to the trabecular space of the vertebra that in turn reinforces and solidifies the target site.

In some instances, an effectiveness of the procedure can be enhanced by forming a cavity or void within the cancellous bone, and then depositing the curable material in the cavity. The cavity can be formed in various manners (e.g., mechanical cutting or shearing of cancellous tissue, expansion of a balloon or other expandable device to compress cancellous bone, etc.). Regardless, to minimize the duration of the procedure and number of tools required, it is desirable to use the same cannula to first deliver the cavity-forming device and subsequently to delivery the curable material. Stated otherwise, one desirable procedure entails initially locating a distal end of the cannula immediately adjacent the target site. The cavity-forming device is then delivered through the cannula and to the target site, and then operated to form the cavity. While the cavity will have an enlarged width (e.g., diameter) as compared to a diameter of the cannula, a smaller width “track” or “dead space” in the cancellous bone between the distal end of the cannula and the cavity normally exists. The cavity-forming device is removed from the cannula, and curable material delivered to the target site via the cannula.

To get the curable material to fill the cavity, the surgeon can either inject the curable material through the cannula and the dead space to reach the cavity, or push the cannula through the dead space until the distal end is in the cavity before delivering the curable material. Under the first approach, curable material is deposited into the dead space, and may undesirably solidify or attach to the cannula itself. Further, the dead space represents an uncontrolled volume that may negatively affect the surgeon's evaluation of whether a necessary volume has been delivered to the cavity. With the second approach, it may be difficult for the surgeon to accurately re-position the cannula within the cavity and/or may cause unintended damage to the tissue surrounding the cavity and/or the cannula itself.

In light of the above, there exists a need in the medical device field for improved systems and methods for delivering stabilizing material to damaged or compromised bone sites.

### SUMMARY

Some aspects in accordance with principles of the present disclosure relate to methods for delivering a material to a surgical target site of a patient. The method includes inserting a distal end of a cannula immediately proximate the target site. The cannula defines a lumen. A portion of a cavity-forming device is extended through the lumen and distally beyond the distal end. The cavity-forming device is then operated to form a cavity at the target site. In this regard, a track is defined in tissue of the target site between the distal end of the cannula and the cavity, with a width of the track being less than a width of the cavity. The cavity-forming device is removed from the cannula, and replaced with a delivery tube. A distal tip of the delivery tube is directed distally beyond the distal end of the cannula, through the track and into the cavity. Finally, a material (e.g., a curable material) is delivered through the delivery tube and into the cavity. With the above techniques, the cannula can remain stationary following initial insertion relative to the target site, and curable material is not directly deposited into the normally occurring “dead space”.

Other aspects in accordance with principles of the present disclosure relate to a system for delivering material into a target site of the patient. The system includes a cannula, a cavity-forming device, a delivery tube, and a source of filling material. The cannula defines a lumen and a distal end. The cavity-forming device includes an elongated body terminating at a distal working end. The elongated body is sized for slidable insertion within the lumen, with the cavity-forming device being configured to form a cavity in tissue of the target site with the working end when the working end is extended distal the cannula. The delivery tube is also sized for slidable insertion within the lumen, and terminates at a distal tip. Finally, the source of filling material is selectively fluidly connected to the delivery tube. With the above construction, the system can be arranged in a cavity-forming state and a material-delivering state. In the cavity-forming state, the elongated body is disposed within the lumen and the working end is distally located a predetermined distance from the distal end of the cannula. In the filling state, the delivery tube is disposed within the lumen and the distal tip is distally located at the predetermined distance from the distal end of the cannula. In some embodiments, the working end of the cavity-forming device includes an inflatable balloon. In other embodiments, the system further includes depth markings or indicators on the elongated body and the delivery tube that establish known positions relative to the cannula. With these embodiments, distal extension of the working end relative to the cannula distal end upon alignment of elongated body depth marking relative to the cannula corresponds with distal extension of the distal tip relative to the cannula distal end upon alignment of the delivery tube depth indicator relative to the cannula.

### BRIEF DESCRIPTION OF THE DRAWINGS

FIG. 1 is a perspective view of a curable material delivery system in accordance with principles of the present disclosure in conjunction with one possible target site;

FIG. 2 is an enlarged side view of cannula assembly and cavity-forming device portions of the system of FIG. 1;

FIG. 3A is a cross-sectional view of the cannula assembly and the cavity-forming device of FIG. 2 in a pre-deployment arrangement;

FIG. 3B is a side view of the cannula assembly and the cavity-forming device in a partial deployment arrangement;

FIGS. 3C and 3D are side views of the cannula assembly and the cavity-forming device in a final deployment arrangement;

FIG. 4 is a simplified side view of an alternative cavity-forming device useful with the system of FIG. 1;

FIG. 5 is a side view of a syringe system useful with the cavity-forming device of FIG. 1;

FIG. 6 is an enlarged side view of the cannula assembly and a delivery tube portion of the system of FIG. 1;

FIG. 7A is a cross-sectional views of the cannula assembly and the delivery tube of FIG. 6 in a first delivery arrangement;

FIG. 7B is an enlarged side view of the cavity-forming device and the delivery tube of the system of FIG. 1, depicting a relationship between depth indicia provided with the two components;

FIG. 7C is a side view of the cannula assembly and the delivery tube in a second delivery arrangement;

FIG. 7D is a side view of the cannula assembly and the delivery tube in a third delivery arrangement;

FIGS. 8A-8C are simplified side views of a portion of the system of FIG. 1 in cavity-forming and delivery states;

FIG. 9A is a simplified plan view of a portion of the curable material delivery system of FIG. 1 employed in a palliative bone procedure in accordance with principles of the present disclosure;

FIGS. 9B-9G are simplified lateral views of a vertebral body, illustrating use of the system in accordance with principles of the present disclosure; and

FIG. 10 is a simplified anterior view of a spinal segment and illustrating use of the system of FIG. 1 in performing another procedure in accordance with principles of the present disclosure.

### DETAILED DESCRIPTION

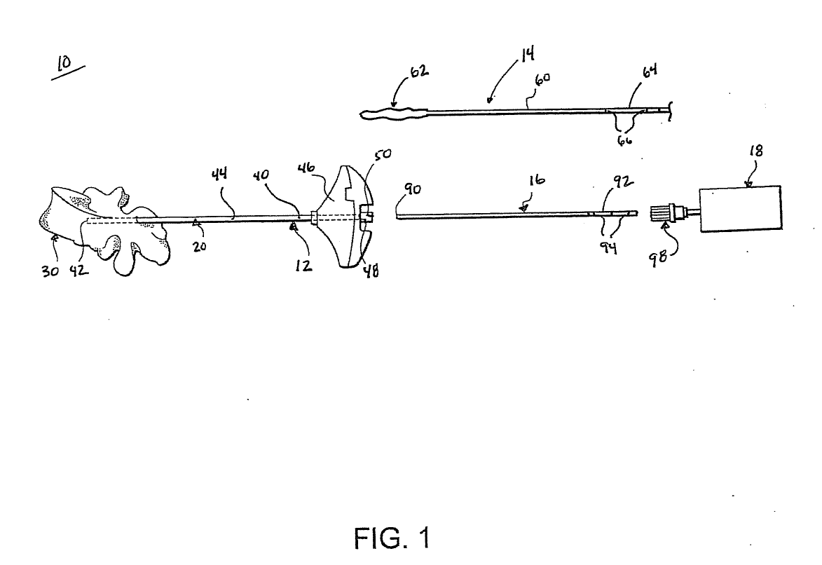

One embodiment of a curable material delivery system 10 in accordance with principles of the present disclosure is shown in FIG. 1. The system 10 includes a cannula assembly 12, a cavity-forming device 14, a delivery tube 16, and a source of curable material 18. Details on the various components are provided below. In general terms, however, the cannula assembly 12 includes a cannula 20 for insertion into a bone site of interest in a patient. In the embodiment depicted in FIG. 1, the bone site of interest is a vertebra 30. Once the cannula 20 is desirably located relative to the bone site 30, a portion of the cavity-forming device 14 is delivered to the bone site 30 via the cannula 20, and operated to form a cavity. The cavity-forming device 14 is then replaced with the delivery tube 16, such that a portion of the delivery tube 16 extends distally beyond the cannula 20 and into the cavity. The curable material source 18 is then operated to deliver curable material to the cavity via the delivery tube 16. The system 10 overcomes the “dead space” issues presented by prior curable material delivery methodologies.

The system 10 can be used for a number of different procedures, including, for example, vertebroplasty and other bone augmentation procedures in which curable material is delivered to a site within bone, as well as possibly to remove or aspirate material from a site within bone. The system 10 is highly useful for delivering a curable material in the form of a bone curable material. The phrase “curable material” within the context of the substance that can be delivered by the system 10 of the present disclosure described herein is intended to refer to materials (e.g., composites, polymers, and the like) that have a fluid or flowable state or phase and a hardened, solid or cured state or phase. Curable materials include, but are not limited to, injectable bone cements (such as polymethylmethacrylate (PMMA) bone curable material), which have a flowable state wherein they can be delivered (e.g., injected) by a cannula to a site and subsequently cure into hardened, cured material. Other materials such as a calcium phosphates, bone-in growth material, antibiotics, proteins, etc., can be used in place of, or to augment, bone cement (but do not affect an overriding characteristic of the resultant formulation having a flowable state and a hardened, solid, or cured state). This would allow the body to reabsorb the curable material and/or improve the clinical outcome based on the type of filler implant material.

As mentioned above, the cannula assembly 12 includes the cannula 20. The cannula 20 is provided to be positioned in (or immediately proximate) a target or injection site for delivery of curable material therein. The cannula 20 is preferably made of a surgical grade of stainless steel, but may be made of known equivalent materials that are both biocompatible and substantially non-compliant at the expected operating pressures. The cannula 20 defines a proximal portion 40, a distal end 42, and a lumen 44 (referenced generally) to allow various equipment, such as the cavity-forming device 14, the delivery tube 16, a stylet (not shown), etc., to pass therethrough. In some embodiments, the distal end 42 is curved or blunt, but can alternatively be beveled to ease the penetration of the cannula 20 through the cutaneous and soft tissues, and especially through hard tissues.

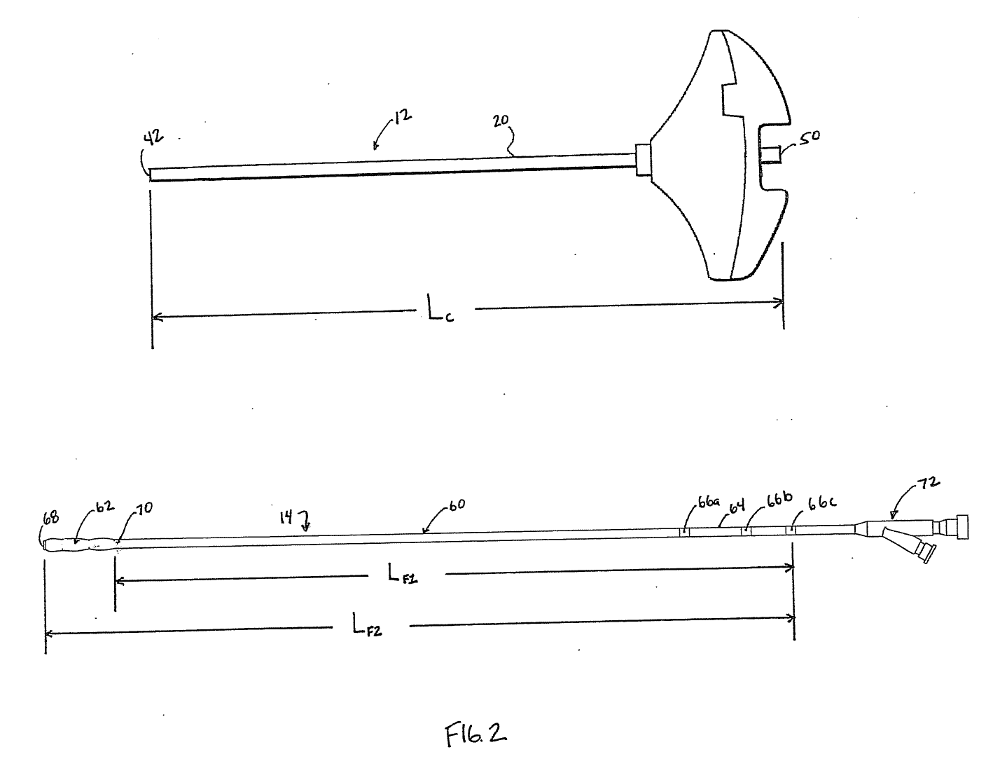

Surrounding the proximal portion 40 of the cannula 20 is an optional handle 46 for manipulating the cannula 20 and connecting the cannula 20 with one or both of the cavity-forming device 14 and/or the delivery tube 16. In some constructions, the cannula assembly 12 further includes a handle connector 48. The handle connector 48 is fluidly connected to the lumen 44, and defines a proximal end 50 of the cannula 20. In some constructions, the handle connector 48 is simply an extension of the cannula 20. In other embodiments, the handle connector 48 can incorporate one or more additional components that are configured to interface with features of the cavity-forming device 14 and/or the delivery tube 16 in establishing a locking mechanism of the system 10. With these optional embodiments, the handle connector 48 can include a luer-lock type of connector, but other known connecting mechanisms may be successfully interchanged, e.g., a conventional threaded hole, a threaded locking nut arrangement, etc. Acceptable examples of the connector/locking mechanism construction are provided in U.S. Publication No. 2007/0198024 entitled “Curable Material Delivery Device” and the teachings of which are incorporated herein by reference. Regardless, a cannula length L_{C} (FIG. 2) is established as a distance between the proximal end 50 and the distal end 42.

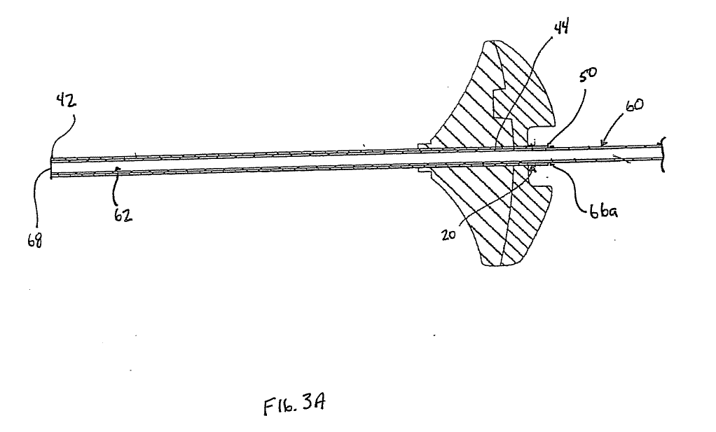

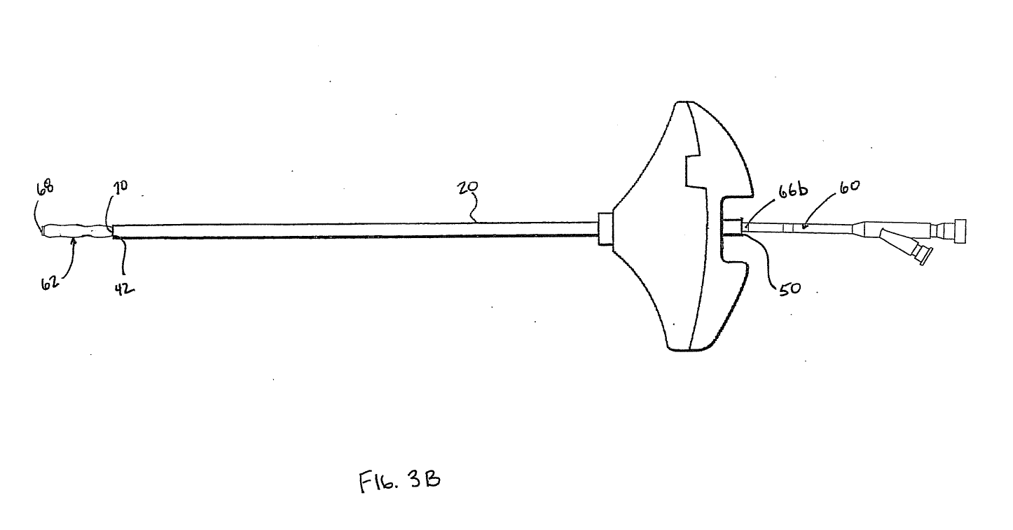

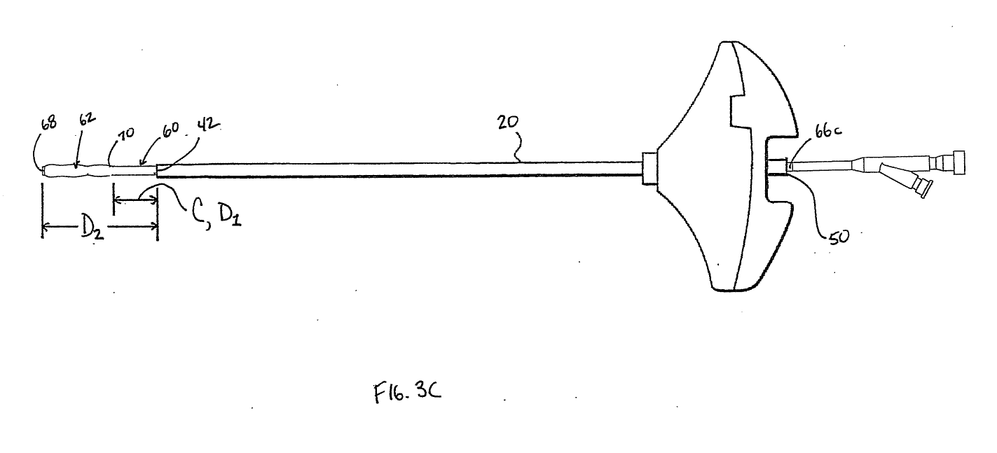

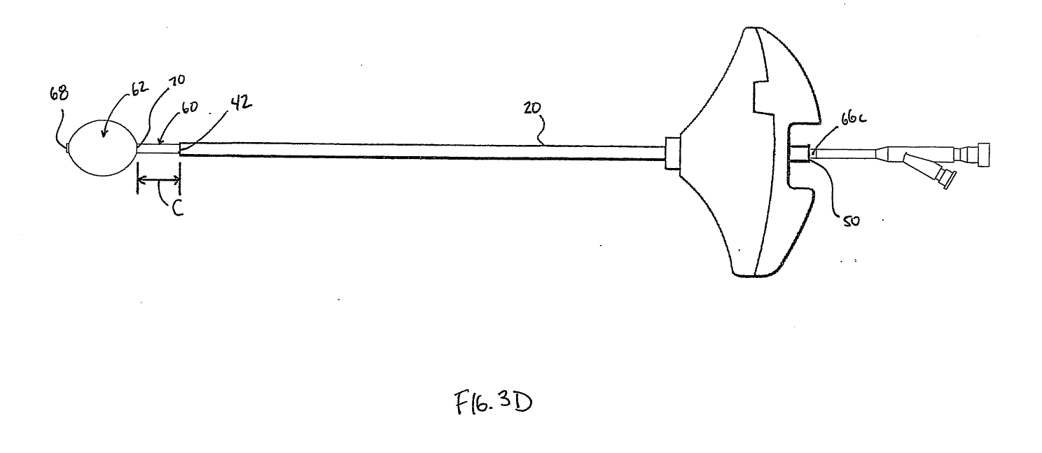

The cavity-forming device 14 can assume various forms appropriate for forming a void or cavity within bone, and generally includes an elongated body 60 distally connected to or forming a working end 62. The elongated body 60 is sized to be slidably inserted within the lumen 44 of the cannula 20, and can include one or more tubes, shafts, etc., necessary for operation of the working end 62. Regardless, a proximal region 64 of the elongated body 60 optionally includes one or more features providing length or depth information. For example, one or more depth markings 66 can be formed along the proximal region 64 as illustrated in FIG. 2. The depth markings 66 are provided at predetermined distances relative to the working end 62, with the distances, in turn, having a predetermined relationship with the cannula length L_{C}. The working end 62 can be described as providing a distal side 68 and a proximal side 70. With this in mind, a first depth marking 66a can be provided at a distance from the distal side 68 corresponding with the cannula length L_{C}. As shown in FIG. 3A, then, when the elongated body 60 is inserted within the cannula lumen 44 and positioned such that the first depth marking 66a (represented schematically in FIG. 3A) is aligned with the proximal end 50 of the cannula 20, the distal side 68 of the working end 62 is immediately proximate the distal end 42 of the cannula 20 (but still “inside” the cannula 20).

As shown in FIGS. 2 and 3B, a second depth marking 66b can also be provided, located at a distance from the distal side 68 corresponding with the cannula length L_{C} plus a length of the working end 62 (i.e., a distance between the proximal side 70 and the second depth marking 66b corresponds with (e.g., is the same as) the cannula length L_{C}). In FIG. 3B, when the second depth marking 66b is aligned with the proximal end 50, the proximal side 70 of the working end 62 is immediately distal the distal end 42 of the cannula 20 (i.e., the working end 62 extends distally from the cannula 20).

In addition, a third depth marking 66c is provided as shown in FIGS. 2 and 3C. The third depth marking 66c is formed at a distance from the distal side 68 corresponding with the cannula length L_{C} plus a length of the working end 62 and a clearance distance C (FIG. 3C) (i.e., a distance between the proximal side 70 and the third depth marking 66c corresponds with (e.g., is the same as) the cannula length L_{C} plus the clearance distance C). The clearance distance C represents a spacing between the proximal side 70 and the cannula distal end 42, and ensures that during operation, the working end 62 will not contact (or be damaged by) the distal end 42 of the cannula distal end 42 as shown in FIG. 3D. For example, where the working end 62 is a balloon, the clearance distance C ensures that with inflation, the working end/balloon 62 will not contact the cannula 20. Because the arrangement of FIG. 3C reflects a desired, final deployment or placement of the working end 62 relative to the cannula distal end 42, the third depth marking 66c can be referred to as the “final deployment depth marking”. While the first and second depth markings 66a, 66b are useful (as well as possibly other depth markings in addition to the final deployment depth marking 66c), in other embodiments, only the final deployment depth marking 66c is included. A hub 72 (shown in FIG. 2) such as a Y-adapter can be provided adjacent the final deployment depth marking 66c (shown in FIG. 2), and in some constructions can serve as or replace the final deployment depth marking 66c (e.g., with these alternative embodiments, when the hub 72 is aligned with the cannula proximal end 50, the working end 62 is at the clearance distance C relative to the cannula distal end 42). In fact, the hub 72 can establish a positive stop or lock with the cannula proximal end 50 at the final deployment depth. Regardless, a cavity subsequently formed by the working end 62 at the final deployment location will have a length approximately extending between the distal side 68 and the proximal side 70. A location of the cavity can thus be defined as having a minimum distance D₁ and a maximum distance D₂ relative to the cannula distal end 42. With these same in-use parameters in mind, an effective working length of the cavity-forming device 14 is established by the final deployment depth marking 66c in a range from a minimum working length L_{F1} at the proximal side 70 to a maximum working length L_{F2} at the distal side 68.

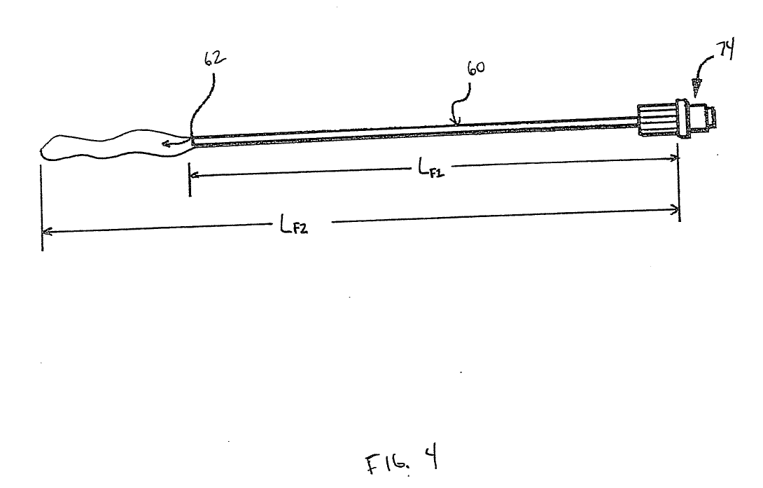

As an alternative (or in addition) to the depth markings 66, the elongated body 62 can be connected to or form a cannula connector 74 as shown in FIG. 4. The cannula connector 74 can assume various forms conducive for selective, rigid attachment to the alternative handle connector 48 (FIG. 1) as described above (e.g., the cannula connector 74 and the handle connector 48 collectively form a locking mechanism), and thus can include or contain a luer-lock threaded fitting. A length of the elongated body 60 between the working end 62 and the cannula connector 74 is predetermined, and is longer than the cannula length L_{C} (FIG. 2). More particularly, the cannula connector 74 is positioned along the elongated body 60 at the minimum and maximum effective working lengths L_{F1}, L_{F2}. With this construction, upon insertion of the elongated body 60 within the lumen 44 and coupling between the cannula connector 74 and the alternative handle connector 48, the working end 62 projects distally beyond the distal end 42 of the cannula 20 at a known or predetermined distal location as described above (i.e., establishes the cavity distances D₁, D₂ relative to the cannula distal end 42 as shown in FIG. 3C).

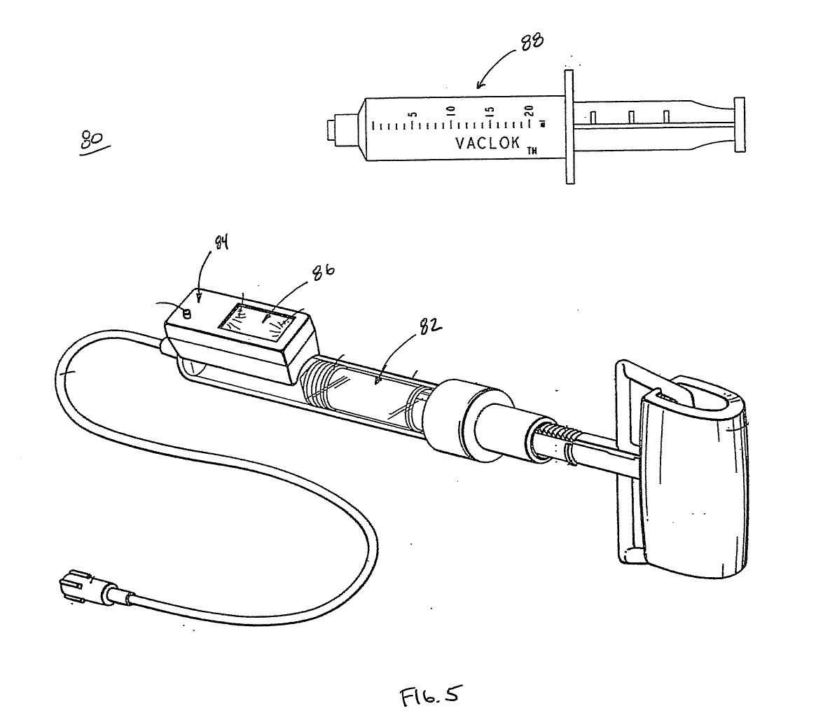

Returning to FIG. 1, the working end 62 can include one or more components appropriate for forming a cavity or void within bone. For example, in some constructions, the working end 62 includes one or more expandable or inflatable members (e.g., a balloon) constructed to transition between a contracted (e.g., deflated) state in which the working end 62 can be passed through the lumen 44 and an expanded (e.g., inflated) state in which the working end 62 expands and compacts contacted cancellous bone. Alternatively, the working end 62 can include a radially expandable cutting-type structure that when exposed distally beyond the confines of the cannula 20 and rotated, impacts and cuts or pulverizes contacted bone. Other cavity-forming configurations, such as ultrasound, thermal, chemical, etc., are also envisioned. In more general terms, then, the working end 62 can have any format that is deliverable through the lumen 44 and operable to form an increased-sized cavity (e.g., radial or width dimension greater than a radius or width of the cannula 20) at a known location relative to the distal end 42 of the cannula 20. Thus, though not shown, the cavity-forming device 14 can include one or more additional components connected or operable through the proximal region 64 of the elongated body 60 for actuating the working end 62. By way of one, non-limiting example, then, the cavity-forming device 14 can include a source (e.g., a manually-operable syringe) of pressurized fluid for inflating one or more balloons carried or formed by the working end 62. For example, FIG. 5 illustrates one embodiment of a syringe system 80 useful in creating pressurized flow of inflation medium. The system 80 includes a primary syringe 82 carrying a display device 84. The display device 84 is electronically connected to a pressure sensor (not shown) located to sense pressure within the syringe 82, and includes a screen 86 (e.g., a distal display) at which the currently sensed pressure is displayed. A memory component (not shown) and related microprocessor (not shown) is optionally further included with the display device 84 and is programmed to store and display additional information at the screen 86, such as the maximum sensed pressure over the course of a particular inflation operation. The maximum sensed pressure can be displayed on the screen at the same time as the currently sensed pressure. Knowing both pressures concurrently is beneficial during a procedure. A secondary syringe 88 can also be included, and employed to prepare the working end/balloon 62 for insertion into the target bone site by removing air from the working end/balloon 62.

Returning to FIG. 1, the delivery tube 16 is sized for insertion within the lumen 44, and defines a distal tip 90 and a proximal section 92. As described below, the delivery tube 16 is employed to deliver curable material. Thus, the delivery tube 16 has an outer diameter that is smaller than a diameter of the lumen 44 of the cannula 20; however, the outer diameter of the delivery tube 16 should not be so small as to allow curable material to readily travel around the outside of the delivery tube 16 and back into the cannula 20. The delivery tube 16 can be formed of any material appropriate for direct contact with the substance to be injected (e.g., bone cement). In some embodiments, the material selected for the delivery tube 16 exhibits minimal bonding with bone cement, such as polypropylene. Alternatively, the delivery tube 16 can be coated with anti-stick material (e.g., silicone). In yet other embodiments, an anti-sticking sheath is disposed over the delivery tube 16 (e.g., the delivery tube 16 is a stainless steel tube, and a polypropylene sheath is applied over the tube 16).

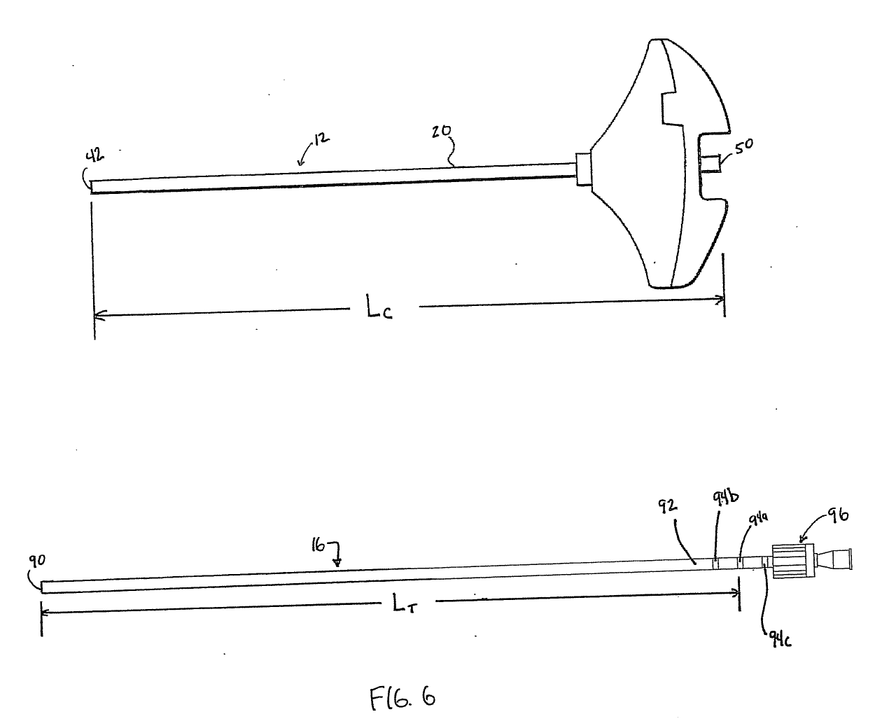

Similar to the cavity-forming device 14, the delivery tube 16 includes, or is provided with, one or more features that provide length or depth information. For example and as best shown in FIG. 6, one or more depth indicators 94 can be formed along the proximal section 92 at distance(s) relating to the cannula length L_{C} and one or both of the cavity distances D₁, D₂ (FIG. 3C) defined by the cavity-forming device 14 relative to the cannula 20 described above, and in particular at distances correlating a location of the distal tip 90 relative to the cannula distal end 42. As described below, locations of the depth indicators 94 relative to the distal tip 90 are directly related to the cavity-forming device minimum and maximum working lengths L_{F1}, L_{F2} (FIG. 2).

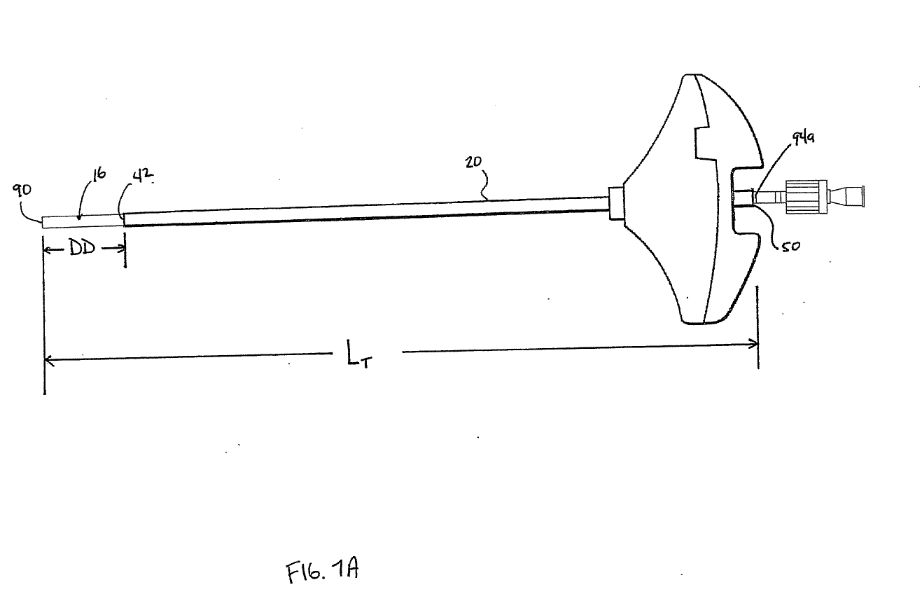

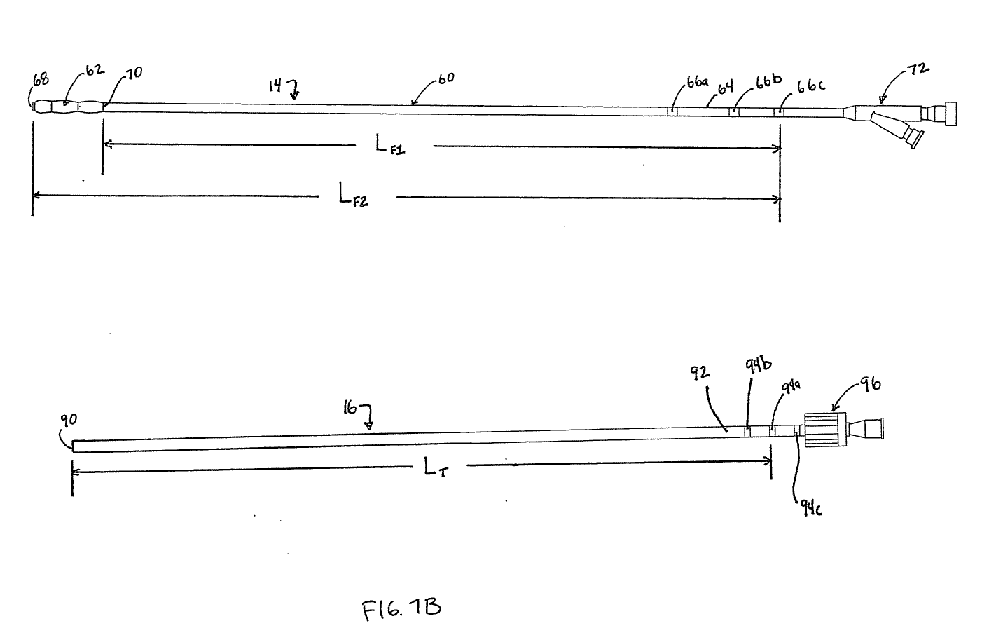

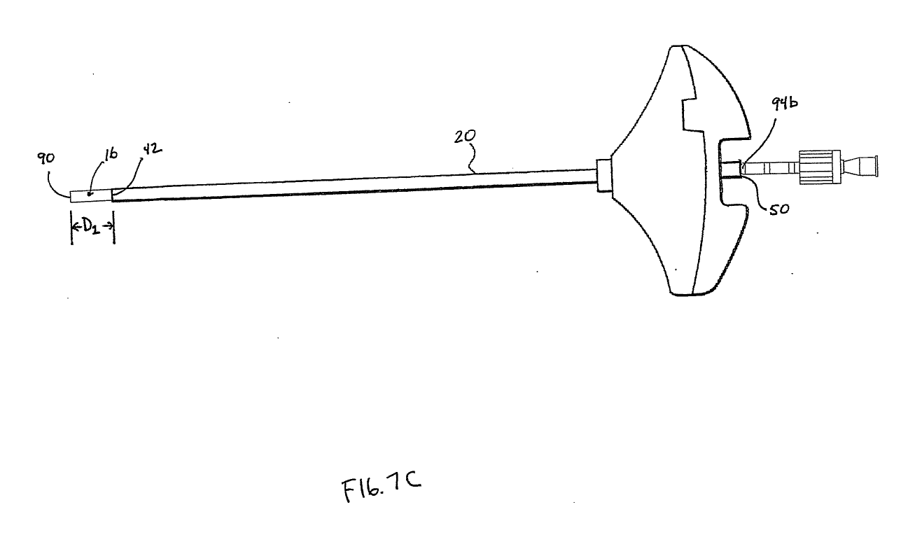

With reference to FIGS. 6 and 7A, a mid-cavity depth indicator 94a can be formed at a distance from the distal tip 90 corresponding with the cannula length L_{C} plus a desired dispensement depth DD. In FIG. 7A, then, when the mid-cavity depth indictor 94a is aligned with the cannula proximal end 50, the distal tip 90 is located at the desired dispensement depth DD relative to the cannula distal end 42. The dispensement depth DD established by the mid-cavity depth indicator 94a reflects a location or depth of the distal tip 90 relative to a cavity formed by the working end 62 (FIG. 3C) and thus is between the minimum and maximum cavity distances D₁, D₂ (FIG. 3C). The mid-cavity depth indicator 94a establishes a delivery tube effective working length L_{T} relative to the distal tip 90 corresponding with in-use location(s) of the working end 62 as described below. Stated otherwise, the delivery tube effective working length L_{T} is within the range of the minimum and maximum cavity-forming device effective working lengths L_{F1}, L_{F2} as illustrated in FIG. 7B.

With reference to FIGS. 6 and 7C, a proximal cavity end depth indicator 94b can be provided. The proximal cavity end depth indicator 94b is formed at a distance from the distal tip 90 corresponding with (e.g., equal to) the cavity-forming device minimum effective working length L_{F1} (FIG. 3C). Thus, when the proximal cavity end depth indicator 94b is aligned with the cannula proximal end 50 (as in FIG. 7C), the distal tip 90 is located at the minimum cavity distance D₁ relative to the cannula distal end 42.

With reference to FIGS. 6 and 7D, a distal cavity end depth indicator 94c can also be provided. The proximal cavity end depth indicator 94c is formed at a distance from the distal tip 90 corresponding with (e.g., equal to) the cavity-forming device maximum effective working length L_{F2} (FIG. 3C). Thus, when the distal cavity end depth indicator 94c is aligned with the cannula proximal end 50 as in FIG. 7D, the distal tip 90 is located at the maximum cavity distance D₂ relative to the cannula distal end 42. Further, a hub 96 (FIG. 6) that serves as an absolute stop to distal movement of the delivery tube 16 relative to the cannula 20 can be included.

As an alternative to the depth indicators 94, the hub 96 is configured as a cannula connector coupled to, or formed by, the proximal section 92 of the delivery tube 16. The cannula connector 96 can be akin to the cannula connector 74 described above (e.g., combines with the handle connector 48 to form a locking mechanism), and thus can assume any of the forms previously described. Regardless, the optional cannula connector format of the hub 96 is configured to selectively, rigidly couple with the handle connector 48, and establishes the predetermined dispensement depth DD (FIG. 7A) upon connection to the handle connector 48.

Returning to FIG. 1, the delivery tube 16 is configured for fluid coupling to the curable material source 18. In some embodiments, a portion of the delivery tube 16 projects proximally beyond the depth indicators 94 (or proximally beyond the optional hub 96), and is fluidly coupled to the curable material source 18, for example via an injection connector 98. Alternatively, auxiliary tubing (not shown) can be provided with the curable material source 18, and fluidly connected to the delivery tube 16 via the optional injection connector 98.

The curable material source 18 can assume various forms appropriate for delivering the desired curable material, and may typically comprise a chamber-filled with a volume of curable material and employ any suitable injection system or pumping mechanism to transmit curable material out of the injector and through the delivery tube 16. Typically, a hand injection system is used where a user applies force by hand to an injector. The force is then translated into pressure on the curable material to flow out of the chamber. A motorized system may also be used to apply force.

The curable material delivery system 10 is arranged in at least a cavity-forming state and a curable material delivery state during use. In the cavity-forming state (FIG. 3C), the cavity-forming device 14 is inserted within the cannula 20, and the final deployment depth marking 66c is aligned with the proximal end 50 of the cannula 20. Alternatively, the connectors 48, 72 can be used to ensure positioning of the working end 62 distally outside of the cannula 20. Regardless, the working end 62 is deployed distal the cannula distal end 42 and is operable to form a cavity.

In the delivery state (FIG. 7A), the cavity-forming device 14 (FIG. 1) is removed from the cannula 20 and replaced with the delivery tube 16 as shown. The distal tip 90 extends or projects distally beyond the distal end of the cannula 20. The desired depth indicator 94 (e.g., the mid-cavity depth indicator 94a) is aligned with the proximal end 50 (or the optional cannula connector utilized to ensure extension of the distal tip 90 beyond the cannula distal end 42) so as to define the delivery tube effective working length L_{T} (FIG. 6), with this working length L_{T} being predetermined and greater than the effective length L_{C} of the cannula 20. The working length L_{T} defines the location (relative to the cannula distal end 42) at which material (e.g., bone cement) is delivered from the delivery tube 16.

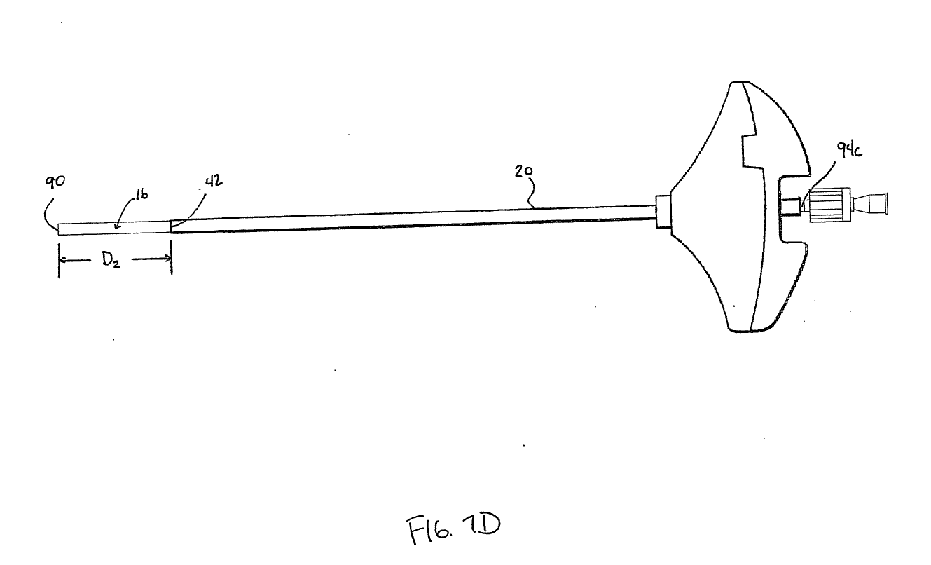

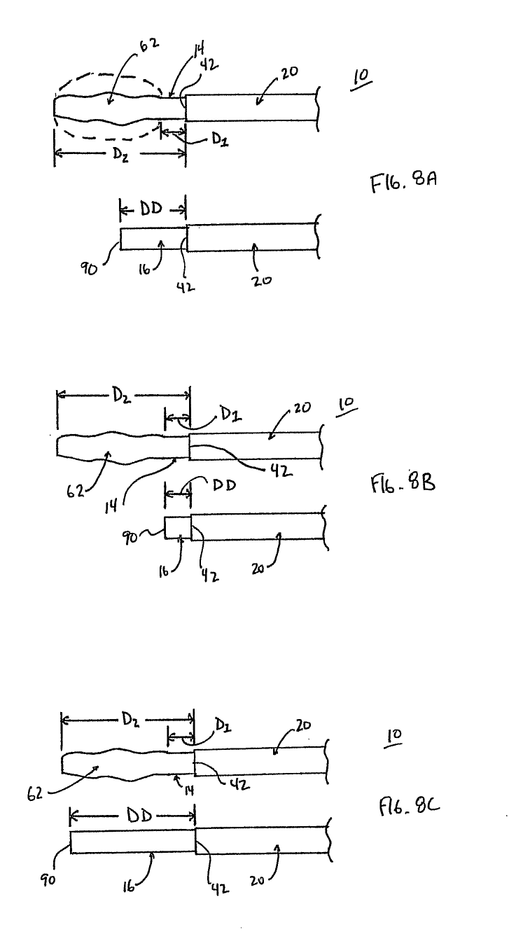

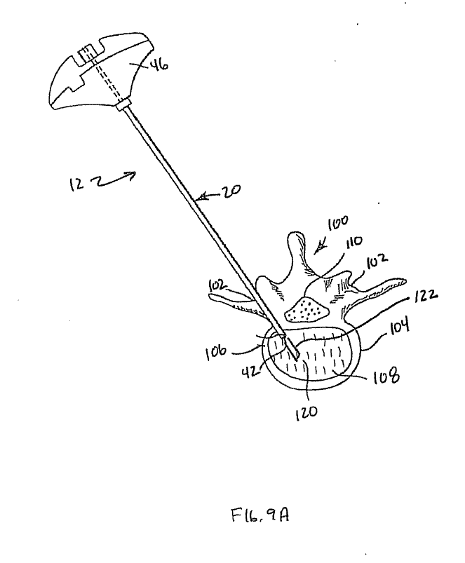

As implicated by the above explanation, correlation between location of the distal tip 90 of the delivery tube 16 relative to the cannula distal end 42 in the delivery state with respect to the predetermined minimum and maximum distances D₁, D₂ defined by the cavity-forming device 14 relative to the cannula distal end 42 in the cavity-forming state can have various forms in accordance with the present disclosure. For example, FIG. 8A illustrates a comparison of one acceptable arrangement of the delivery system 10 in the cavity-forming and delivery states. In particular, a distal location of the distal tip 90 relative to the distal end 42 of the cannula 20 (i.e., the dispensement depth DD) is approximately at a midpoint of the distal location of the working end 62 relative to the distal end 42 of the cannula 20 (i.e., the mid-point of the cavity minimum and maximum distances D₁, D₂). This can be achieved, for example, by aligning the mid-cavity depth indicator 94a (FIG. 6) with the cannula proximal end 50 (FIG. 7B). As a point of reference, FIG. 8A further illustrates, with dashed lines, an arrangement of the working end 62 in an inflated state.

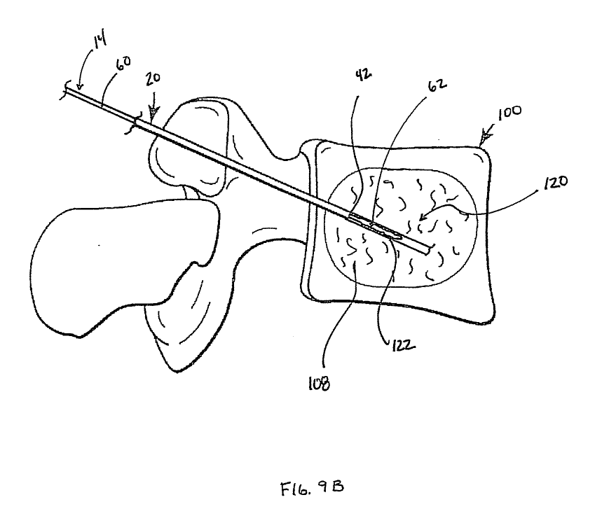

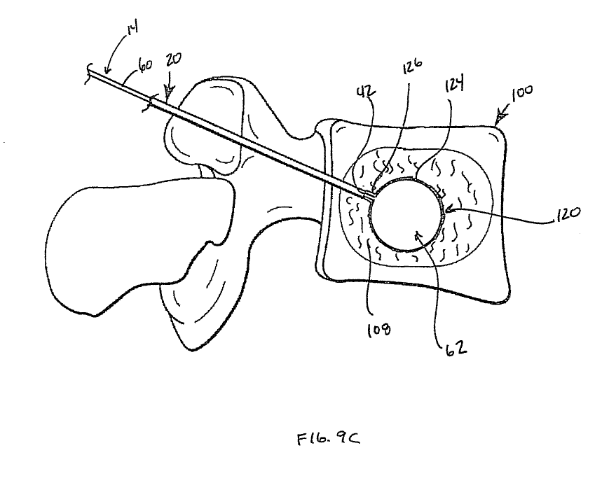

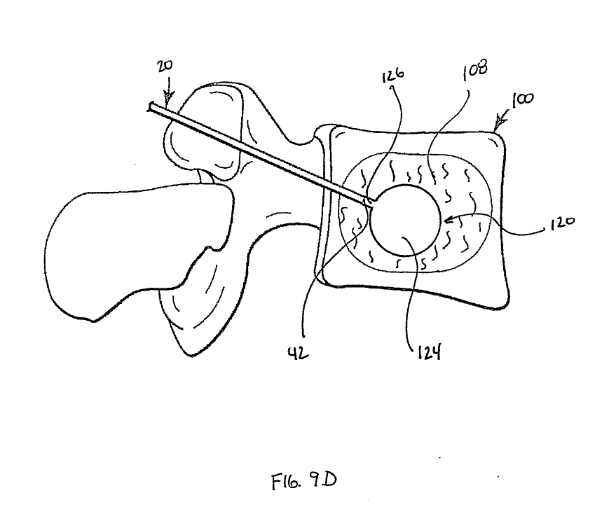

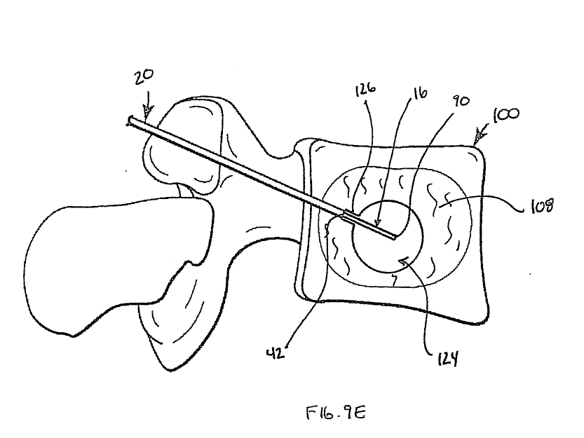

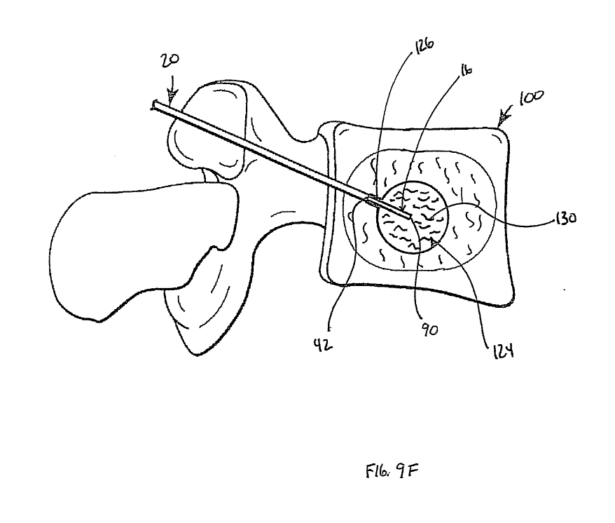

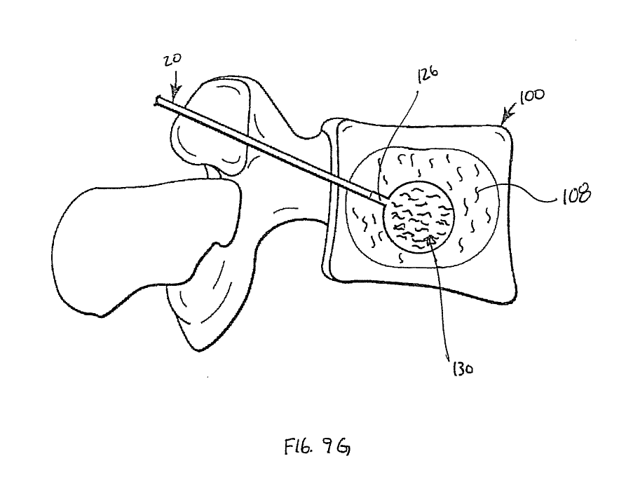

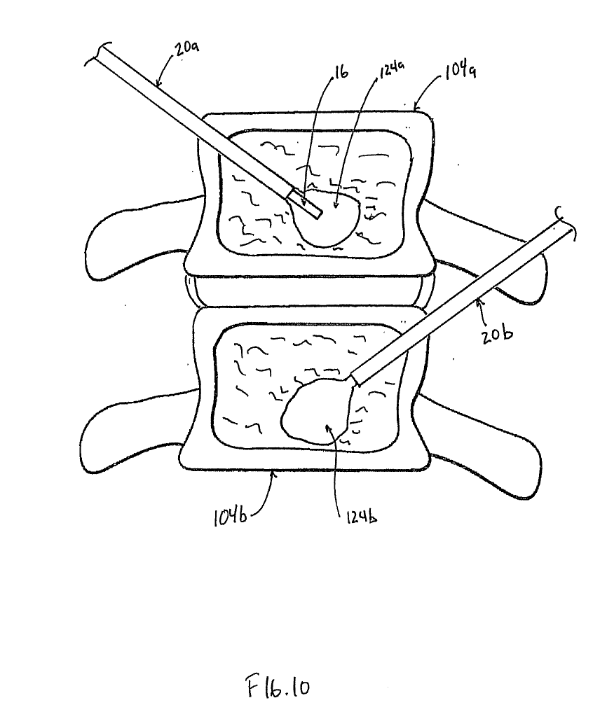

In FIG. 8B, the predetermined dispensement depth DD of the distal tip 90 relative to the cannula distal end 42 approximates the minimum cavity distance D₁ defined by the proximal side 70 of the working end 62 relative to the cannula distal end 42. This can be achieved, for example, by aligning the proximal cavity end depth indicator 94b (FIG. 6) with the cannula proximal end 50. In FIG. 9C, the predetermined dispensement depth DD of the distal tip 90 relative to the cannula distal end 42 approximates the maximum cavity distance D₂ defined by the distal side 68 of the working end 62 relative to the cannula distal end 42. This can be achieved, for example, by aligning the distal cavity end depth indicator 94c (FIG. 6) with the cannula proximal end 50. In more general terms, then, the predetermined dispensement depth DD established by one or more of the depth indicators 94 can have any relationship that locates the distal tip 90 in a region affected by operation of the working end 62 in instances where the cannula 20 remains stationary and the cavity-forming device 14 is replaced with the delivery tube 16. Along these same lines, in some embodiments the delivery tube 16 can be selectively repositionable between the locations of FIGS. 8A-8C in the delivery state.

Regardless of an exact configuration, the curable material delivery system 10 in accordance with principles of the present disclosure is highly useful in performing a wide variety of bone stabilizing procedures as part of an overall curable material delivery procedure. To this end, FIG. 9A illustrates use of the system 10 in delivering curable material into a target site of a vertebra 100. In general terms, the vertebra 100 includes pedicles 102 and a vertebral body 104 defining a vertebral wall 106 surrounding bodily material (e.g., cancellous bone, blood, marrow, and soft tissue) 108. The pedicles 102 extend from the vertebral body 104 and surround a vertebral foramen 110. As a point of reference, systems of the present disclosure are suitable for accessing a variety of bone sites. Thus, while the vertebra 100 target site is illustrated, it is to be understood other that bone sites can be accessed by the system 10 (i.e., femur, long bones, ribs, sacrum, etc.).

The cannula 20 is initially employed to form an access path to a target site 120, for example through one of the pedicles 102 and into the bodily material 108. Thus, as illustrated, the cannula 20 has been driven through the pedicle 102 via a transpedicular approach. The transpedicular approach locates the cannula 20 between the mammillary process and the accessory process of the selected pedicle 102. Alternatively, other approaches to the target site 120 can be employed (e.g., anterior). In any event, the cannula 20 provides access to the target site 120 at the open, distal end 42. One or more stylets (not shown) can be employed to assist in forming an access channel 122 to the target site 120. For example, a series of differently-sized or configured stylets (e.g., sharp ended or blunt) can be sequentially deployed through the cannula 20 to form the channel 122. Alternatively, or in addition, an outer guide cannula (not shown) can initially be deployed to form an access path for insertion of the cannula 20. Regardless, once positioned, the cannula 20 can remain relatively stationary relative to the target site 120.

Once the cannula 20 is positioned within the bodily material 108 at the desired target site 120, the cavity-forming device 14 is assembled to the cannula 20. For example, as shown in greater detail in FIG. 9B, the elongated body 60 is slidably inserted within the cannula 20, with the working end 62 being distally advanced therethrough. Upon alignment of the final deployment depth marking 66c (FIG. 3C) with the proximal end 50 (FIG. 1) of the cannula 20, the working end 62 is distal the distal end 42 of the cannula 20, and is positioned at the target site 120. In this regard, FIG. 9B reflects the channel 122 being defined within the bodily material 108, and the working end 62 having been passed through or within the channel 122. The channel 122 can be created during insertion of the working end 62 into the bodily material 108, or can be formed by the cannula 20 or other component (e.g., a stylet (not shown)) prior to deployment of the cavity-forming device 14 as described above. Regardless, the cavity-forming device 14 is operated to cause the working end 62 to form a cavity or void 124 in the bodily material 108 (e.g., the working end 62 is expanded) as shown in FIG. 9C. The cavity 124 can have a variety of different shapes differing from that implicated by FIG. 9C.

Following formation of the cavity 124, the cavity-forming device 14 is transitioned to a contracted state, and withdrawn from the target site 120 and the cannula 20. FIG. 9D reflects the cavity 124 upon removal of the cavity-forming device 14 from the cannula 20. As shown, a small track segment 126 remains, extending between and interconnecting the distal end 42 of the cannula 20 and the cavity 124. As a point of reference, the cavity 124 can generally be described as having or defining a width or other dimension) (e.g., diameter perpendicular to an axis of the cannula 20 that is greater than a diameter of the cannula 20, whereas the track segment 126 is substantially smaller in width (or other corresponding dimension than the cannula diameter). The cannula 20, and in particular the distal end 42, can remain stationary relative to the target site 120 as the cavity-forming device 14 is withdrawn.

With the cannula 20 still in the same location relative to the target site 120, the delivery tube 16 is then inserted into the cannula 20 and advanced to the target site 120, and in particular within the cavity 124, as shown in FIG. 9E. Alignment of the mid-cavity depth indicator 94a (FIG. 7B) with the proximal end 50 (FIG. 1) of the cannula 20 ensures that the distal tip 90 of the delivery tube 16 is positioned within the target site 120 (and in particular the cavity 124) as described above. As a point of reference, while the distal tip 90 is illustrated as being approximately centrally located within the cavity 124, a more distal or more proximal arrangement (within the cavity 124) is also envisioned. For example, where provided, the proximal cavity end depth indicator 94b (FIG. 6) or the distal cavity end depth indicator 94c (FIG. 6) can be utilized to position the distal tip 90 at the proximal side or distal side, respectively, of the cavity 124.

The curable material source 18 (FIG. 1) is then operated to deliver curable material 130 into the cavity 124 via the delivery tube 16 as shown in FIG. 9F. The delivery tube 16 essentially occupies the track segment 126, thereby preventing unnecessary distribution of the curable material 130 into the track segment 126. In some embodiments, the delivery tube 16 remains in the position of FIG. 9F during the entire delivery procedure; in other embodiments, the distal tip 90 can be proximally retracted (or distally extended) within the cavity 124 while delivering the curable material 130. For example, the distal tip 90 can be sequentially retracted while the curable material 130 is delivered to better ensure complete filing of the cavity 124, with the optional proximal cavity end depth indicator 94b (FIG. 6) providing a visual “warning” of maximum retraction (e.g., once the user sees the proximal cavity end depth indicator 94b at the cannula proximal end 50 (FIG. 1), s/he understands that the distal tip 90 is in close proximity to the track segment 126 and that proximal refraction of the delivery tube 16 should cease). Once a desired volume of the curable material 130 has been delivered to the target site 120, the delivery tube 16, as well as the cannula 20, are removed from the patient. The now-stabilized vertebra 100 (following removal of the delivery tube 16) is illustrated in FIG. 9G.

Yet another procedure envisioned by the present disclosure can be described with reference to FIG. 10 and includes delivering curable material to two (or more) target sites. For example, FIG. 10 illustrates first and second vertebral bodies 104a, 104b. First and second cannulas 20a, 20b have been delivered to the vertebral bodies 104a, 104b, respectively, as described above, followed by formation of a cavity 124a, 124b in each body 104a, 104b, respectively. The delivery tube 16 is employed to deliver curable material to the cavity of the first vertebral body 104a via the first cannula 20a. Once a desired volume has been delivered, the delivery tube 16 is removed from the first cannula 20a, inserted into the second cannula 20b, and employed to deliver curable material to the cavity of the second vertebral body 104b. With this approach the delivery tube 16 is effectively “pre-filled” with curable material upon removal from the first cannula 20a and insertion into the second cannula 20b, thus reducing an overall time required to complete the procedure.

As an additional advantage, when the delivery tube 16 is removed from the first cannula 20a (and still “filled” with the curable material), it can be temporarily stored at a location in the surgical suite that is outside of the patient (and likely at room temperature). Because curable materials commonly employed for bone augmentation (e.g., bone cement) are formulated to harden or set at body temperature, by temporarily storing the “pre-filled” delivery tube 16 outside of the patient's body (and at a temperature lower than body temperature), the surgeon has extra time to perform the next curable material delivery operation. In other words, maintaining the delivery tube 16 at a temperature lower than body temperature affords the surgeon more time before hardening of curable material with the delivery tube occurs as compared to a technique in which the delivery tube 16 is held within the patient's body between delivery operations.

Systems and methods in accordance with the present disclosure provided a marked improvement over previous designs. The distally-extending delivery tube eliminates the “dead space” issues attendant with previous designs. Further, by optionally filling the cavity from an anterior position, extravasations can be avoided, and no “wasted” curable material fills the cannula.

Although the present disclosure has been described with reference to preferred embodiments, workers skilled in the art will recognize that changes can be made in form and detail without departing from the spirit and scope of the present disclosure.

What is claimed is:

1. A method for delivering material to a surgical target site of a patient, the method comprising:
inserting a distal end of a cannula immediately proximate the target site, the cannula defining a lumen;
extending a portion of a cavity-forming device through the lumen and distally beyond the distal end;
operating the cavity-forming device to form a cavity at the target site, wherein a track is defined in tissue of the target site between the distal end of the cannula and the cavity, a width, such as a diameter, of the track being less than or equal to a width of the cavity;
removing the cavity-forming device from the cannula,
inserting a delivery tube into the lumen;
directing a distal tip of the delivery tube distally beyond the distal end of the cannula, through the track, and into the cavity; and
delivering material through the delivery tube and into the cavity.

2. The method of claim 1, wherein the distal end of the cannula remains relatively stationary during the steps of operating the cavity-forming device, removing the cavity-forming device, inserting the delivery tube, and directing the distal tip of the delivery tube into the cavity.

3. The method of claim 1, wherein the method is characterized by an absence of material being dispensed into the track.

4. The method of claim 1, wherein the target site is within a vertebra.

5. The method of claim 1, wherein the material is a curable material.

6. The method of claim 1, further comprising:
inserting a stylet through the lumen and distally beyond the distal end to form a channel prior to the step of extending a portion of a cavity-forming device through the lumen.

7. The method of claim 6, wherein the portion of the cavity-forming device is inserted within the channel.

8. The method of claim 1, wherein the cavity-forming device includes an inflatable balloon, and further wherein the step of operating the cavity-forming device to form a cavity includes inflating the balloon.

9. The method of claim 1, wherein the step of delivering material includes:
proximally retracting the distal tip within the cavity while delivering the material.

10. The method of claim 1, wherein the delivery tube includes a first cavity depth indicator, and further wherein the step of delivering the distal tip into the cavity includes aligning the first cavity depth indicator with a proximal end of the cannula.

11. The method of claim 10, wherein the delivery tube includes a second cavity depth indicator located distal the first cavity depth indicator, and further wherein the step of delivering material includes proximally retracting the delivery tube relative to the cannula until the second cavity depth indicator is aligned with the proximal end of the cannula.

12. A system for delivering material into a target site of a patient, the system comprising:
a cannula defining a lumen and a distal end;
a cavity-forming device including an elongated body terminating at a distal working end, wherein the elongated body is sized for insertion within the lumen and the cavity-forming device is configured to form a cavity in tissue of the target site with the working end when the working end is extended distal the distal end of the cannula;
a delivery tube sized for slidable insertion within the lumen and terminating at distal tip; and
a source of filling material fluidly connected to the delivery tube;
wherein the system is operable in a cavity-forming state in which the elongated body is disposed within the lumen and the working end is distally located a predetermined distance from the distal end, and a filling state in which the delivery tube is disposed within the lumen and the distal tip is distally located at the predetermined distance from the distal end.

13. The system of claim 12, further comprising a polypropylene sheath disposed over the delivery tube.

14. The system of claim 12, wherein the working end includes an inflatable balloon.

15. The system of claim 14, wherein the balloon includes a proximal side connected to the elongated body and a distal side opposite the proximal side, and further wherein the predetermined distance is between the proximal and distal sides.

16. The system of claim 12, further comprising a depth marking on the elongated body and a depth indicator on the delivery tube, wherein distal extension of the working end relative to the distal end of the cannula upon alignment of the depth marking relative to the cannula corresponds with distal extension of the distal tip relative to the distal end of the cannula upon alignment of the depth indicator relative to the cannula.

17. The system of claim 12, further comprising:
an inflation device for delivering an inflation medium into the working end, the inflation device including a syringe and a display device programmed to display a sensed pressure of the syringe.
